SEQUENCE LISTING (7 sequences)

SEQ ID NO: 1 - protein, 231 aa, Artificial
  Fc receptor binding protein
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKGRWQQGNVFSCSVMHEALHNHYTQKSLSLSPGK

SEQ ID NO: 2 - protein, 231 aa, Artificial
  Fc receptor binding protein
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKGRWQQGNVFSCSVMHEALHNHYTQKSLSLSPGK

SEQ ID NO: 3 - protein, 231 aa, Homo sapiens
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 36 bp, Artificial
  forward primer for amplification of external domain of HA3 gene
gcgcggccattatggcccaaaaccttcccggaaatg

SEQ ID NO: 5 - DNA, 36 bp, Artificial
  reverse primer for amplification of external domain of HA3 gene
gcgcggccgaggcggccccagtctttgtatcctgac

SEQ ID NO: 6 - DNA, 36 bp, Artificial
  forward primer for amplification of external domain of HA from H5 avian influenza virus A
gcgcggccattatggccaagatcagatttgcattgg

SEQ ID NO: 7 - DNA, 34 bp, Artificial
  reverse primer for amplification of external domain of HA from H5 avian influenza virus A
gcgcggccgaggcggccttggtaaattcctattg

(19) United States
(12) Patent Application Publication
(10) Pub. No.: US 2012/0225067 A1
(43) Pub. Date: Sep. 6, 2012

(54) Composition and Method for Mediating an Immune Response

(75) Inventor: CAMILO COLACO, CAMBRIDGE (GB)

(73) Assignee: IMMUNOBIOLOGY LTD, CAMBRIDGE (GB)

(21) Appl. No.: 13/470,227

(22) Filed: May 11, 2012

Related U.S. Application Data
(63) Continuation of application No. 12/225,454, filed on Sep. 19, 2008, now abandoned.

(30) Foreign Application Priority Data
Mar. 22, 2006 (GB) 0605735.0

(51) Int. Cl. A61K 39/395; A61P 37/04; A61P 25/28; A61P 31/04; A61P 31/12; A61P 35/00; C07K 16/46; A61P 31/00

(52) U.S. Cl. 424/134.1; 530/387.3

(57) ABSTRACT

The present invention provides a fusion protein comprising an Fc receptor binding polypeptide and an antigenic polypeptide. The fusion protein may further comprise a linker sequence or hinge portion which joins the Fc receptor binding polypeptide and the antigenic polypeptide. The Fc receptor binding polypeptide typically comprises the CH2 constant domain of a human IgG immunoglobulin. The antigenic polypeptide can be any polypeptide which induces an immune response. Administration of the fusion protein to a subject results in a cytotoxic T lymphocyte response being induced against the antigenic polypeptide provided within the fusion protein. The invention further extends to methods for the treatment of a disease condition in a subject using the fusion proteins of the invention.

### FIELD OF THE INVENTION

The present invention provides an immunoconjugate for use in mediating an immune response in a host. More specifically, there is provided a fusion protein which comprises an Fc receptor binding polypeptide which, upon binding an Fc receptor, causes internalisation of the Fc receptor and the associated bound fusion protein. The fusion protein has a particular utility in methods for the treatment and prophylaxis of infection caused by a pathogenic organism, in particular viral pathogens.

### BACKGROUND OF THE INVENTION

The adaptive immune response is mediated by two complimentary mechanisms, humoral involving soluble molecules especially immunoglobulins and cellular involving lymphocytes especially T-cells.

The Fc domains of immunoglobulins have been shown to have effector functions which are primarily complement fixation and Fc receptor (FcR) binding. Fc receptors bind to the constant domains of immunoglobulins and a number of receptors have been defined that are thought to mediate accessory functions including opsonisation and ADCC (Daeron 1997, M. Annual Review Immunology, 15; 203-234, Ravetch and Clynes, Annual Review of Immunology. 1998. 16:421-432). Different Fc receptors, when bound, can mediate different downstream signaling. More particularly, some Fc receptors, when bound, have been shown to activate an immune response, while other Fc receptors inhibit an immune response. Thus FcRs have been shown to be either activating or inhibitory for the immune response depending on the presence of activating (ITAM) or inhibiting (ITIM) motifs in their cytoplasmic domains (Daeron, M. Annual Review Immunology, 15; 203-234. 1997).

Previous patents have sought to activate the immune response by targeting antigens to activating FcRs through the use of bifunctional agents containing antibodies against FcRs (see for example WO 96/40788). These reagents bind to the FcR but do not induce their internalisation, and hence associated processing and antigen presentation (T. Keler et. al. 2002 J Immunol. 165: 6738-6742). The internalisation of an Fc receptor is induced by the binding of an immunoglobulin to the FcR via the Fc domain in instances where the binding occurs with a sufficiently high avidity (A. Yada et al. 2003 Cell Immunol 225(1):p 21-32, P. T. Harrison et al. 1994 J Biol Chem. 269 (39): 24396-24402). It is though that this binding interaction can be mimicked by the use of modified intact immunoglobulin containing foreign sequences introduced into the variable domains by CDR-grafting (WO 02/058728) but it is unclear whether the Fc domain is necessary and/or sufficient to induce the internalisation of FcRs (P. T. Harrison et al. 1994 J Biol Chem. 269 (39): 24396-24402, A Yada et al. 2003 Cell Immunol 225(1):p 21-32). Moreover, it is unclear as to whether the Fc domain is itself sufficient to induce the internalisation of FcRs. In fact the Fc domain has been extensively used to prolong the circulatory half-life of therapeutic proteins with the development of numerous Fc-fusion proteins for cytokines and other immunomodulators such as the TNFR-Fc fusion protein Etanercept (ENBREL™).

Following extensive experimental research and innovative thinking, the inventor has identified the factors required for internalisation of the FcR following ligand binding. The fusion protein of the present invention comprises an antigenic polypeptide sequence and an FcR binding polypeptide. Moreover the fusion proteins of the present invention induce an immune response against the antigenic sequence. The FcR binding polypeptide provided in the fusion protein is selected so that it will bind to the Fc receptor with sufficient binding affinity that internalisation of the Fc receptor and bound fusion protein will occur. The internalisation of the FcR/immunoconjugate complex allows the immunoconjugate to be processed by the cell's antigen processing pathway. Antigen processing causes the immunoconjugate to be broken down into fragments, with these fragments being presented to the cells of the immune system.

When internalisation of a bound Fc receptor is not induced, ligand binding merely results in intracellular signalling mediated by the cytosolic portion of the Fc receptor. Although this signalling can contribute to an immune response, it is not sufficient to cause internalisation of the FcR, thus processing and presentation of any antigenic peptide complexed to the FcR ligand does not occur.

Influenza viruses are orthomyxoviruses, which fall into three types; A, B and C. Influenza A viruses can be divided into subtypes according to their surface proteins, haemagglutinin (HA or H) and neuraminidase (NA or N). There are 14 known H subtypes and 9 known N subtypes. All H subtypes have been found in birds, however only three H subtypes (H1, H2 and H3) and two N subtypes (N1 and N2) have been reported as commonly circulating in humans.

Seasonal influenza epidemics in humans are associated with amino acid changes in antigenic sites in the haemagglutinin and neuraminidase proteins in a process termed ‘antigenic drift’. Major pandemics are associated with the introduction of new haemagglutinin and neuraminidase genes from animal-derived influenza viruses, by reassortment, into the genetic background of a currently circulating human virus in a process termed ‘antigenic shift’.

At present, there is no vaccine against H₅N₁ for use in humans. According to the WHO, vaccines in development against the 2003 strain of H5N1 are not protective against the 2004 Vietnam H5N1 strain. Vaccine development for human therapeutic use will not be possible until a human-to-human transmissible strain emerges, and this will take a number of months to be ready for wide scale administration. Accordingly, preventing an influenza pandemic through the use of vaccines derived from an infectious strain is not a reliable means for the control of disease spread. Therefore, the development of a broad-spectrum therapeutic to prevent and treat infection from multiple influenza strains and subtypes would be highly desirable.

The inventor of the present invention has surprisingly provided an Fc protein-antigen fusion protein immunoconjugate which can mediate long term protective immunity against the pathogen from which the antigen portion of the immunoconjugate is derived. The immunity mediated against the pathogen is induced by means of a cytotoxic T lymphocyte (CTL) response, wherein the response which is mediated surprisingly lacking in a significant neutralising antibody response component. This is significant, as it is generally the humoral element of an immune response which is considered essential in order for a vaccine to mediate long term protective immunity in a subject to whom it is administered.

The fusion protein of the invention can surprisingly induce an immune response which provides a subject with a protective immunity to not only a specific strain of a pathogen, but also to further related strains of the pathogen which may result from, for example, mutation or antigenic drift.

Surprisingly, this immunity is substantially conferred without a significant humoral response being mounted by the subject to whom the fusion protein immunoconjugate of the invention is administered, and accordingly antibody production and seroconversion do not substantially contribute to the long term protective immunity in a subject against a pathogen. The inventor has identified that the immunoconjugate has a specific utility in mediating long term protective immunity against infectious diseases, for example disease conditions mediated by viral pathogens such as HIV and, in particular, influenza, where events such as antigenic shift and antigenic drift cause variance in the infectious agent which causes the disease. Moreover, the immunoconjugate also has a specific utility in the induction of immune responses against pathogens that show high rates of sequence mutation and variation.

### SUMMARY OF THE INVENTION

According to a first aspect of the present invention there is provided a fusion protein comprising one or more antigenic polypeptide sequences and an Fc receptor binding polypeptide which binds to an Fc receptor with a binding affinity sufficient to cause internalisation of the bound Fc receptor.

By the term “a binding affinity sufficient to cause internalisation of the bound Fc receptor”, it is meant that the Fc receptor is bound by a ligand with a binding affinity which causes the bound complex to be internalised within the cell. This results in the bound fusion protein of the invention being processed by the cell's antigen processing pathway. This results, in turn, in the fusion protein being broken down and displayed on the surface of the cell by means of major histocompatability molecules (MHC), these presenting the peptide fragments to the cells of the immune system.

As defined herein, the term ‘internalisation’ means endocytosis and in particular, receptor mediated endocytosis or Fc mediated phagocytosis. Endocytosis is the process of the uptake of macromolecules or particles into cells. In relation to the present invention, internalisation particularly occurs when an Fc receptor is bound by a ligand with a sufficient affinity that internalisation of the Fc receptor and the bound ligand occurs.

In one embodiment, in order to induce internalisation, typically the Fc receptor binding polypeptide of the fusion protein will be required to bind to the Fc receptor with a dissociation rate constant binding affinity of from about 10⁻⁶ Kd to about 10⁻⁹ Kd. In a further embodiment the binding affinity between the Fc receptor binding polypeptide and the Fc receptor is from about 10⁻⁸ Kd to about 10⁻⁹ Kd. In a further embodiment, the association rate constant between the Fc receptor binding polypeptide of the fusion protein of the invention and the Fc receptor will be in the region of from about 1×10⁶ Ka to about 3×10⁹ Ka.

In certain embodiments the Fc receptor binding protein is derived from the heavy chain of an immunoglobulin, typically a human immunoglobulin. Typically the immunoglobulin is the human immunoglobulin IgG, and accordingly the Fc receptor binding protein can be derived from the heavy chain of human IgG, in particular from the heavy chain of IgG subclass IgG1 or the heavy chain of IgG subclass IgG3.

In certain further embodiments, the Fc receptor binding protein comprises the CH2 constant domain of the IgG1 human immunoglobulin or the CH2 constant domain of the IgG3 human immunoglobulin. The CH2 constant domain of human IgG1 or human IgG3 is also known as the Cγ2 domain or the C2 domain, this alternative nomenclature being derived from the heavy chain of human IgG being designated the γ chain.

In further embodiments, particularly where the Fc receptor binding protein is derived from the human IgG1 immunoglobulin or the human IgG3 immunoglobulin, the sequence of the Cγ2 domain present within the Fc receptor binding protein retains a highly-conserved N-linked glycosylation site at the asparagine (Asn, N) residue at position 297 (N297). The glycosylation of this residue has been identified as being important for mediating high affinity binding and activation of Fc receptors. Without wishing to be bound by theory, the inventor predicts that the presence of the glycosylated residue at residue 297 of the C2 constant domain of the IgG heavy chain contributes to the tertiary structure which is important in allowing binding to the Fc receptor. In particular, it is predicted that further amino acid residues which are present in the CH2 constant domain sequence, or fragment thereof, which may be present within the Fc receptor binding polypeptide of the fusion protein fold around the N297 glycosylation site, this causing the N297 residue to be located within or substantially encapsulated by the adjacent residues. This folding results in a tertiary structure which can be bound by an Fc receptor. Accordingly, the presence of a glycosylated residue at this position is important in serving as a framework which allows further amino acids in the Fc receptor binding polypeptide to fold thereabout. The absence of a glycosylated residue at the position, or at a residue equivalent to this position in a synthetic sequence affects Fc receptor binding.

In a further embodiment, site directed mutagenesis may be performed on the polynucleotide sequence encoding the Fc receptor binding polypeptide. This mutagenesis serves to alter, by substitution, addition or deletion, the coding sequence. Changes in the resulting polynucleotide sequence can alter the affinity of Fc receptor binding. The invention therefore further extends to an Fc receptor binding protein which comprises a Cγ2 domain derived from a human IgG immunoglobulin, typically an IgG immunoglobulin of the subclass IgG1 or IgG3, which has been subjected to at least one mutation in its amino acid sequence. For example, the mutation may relate to the replacement of the serine residue at position 298 by an alanine residue and/or the replacement of the glutamine residue at position 295 by an alanine residue. Where a CH2 domain sequence is provided with specific mutations, the polynucleotide may be encoded from a synthetic gene construct.

Accordingly in various further embodiments the Fc receptor binding polypeptide comprises the CH2 constant domain or a fragment thereof which comprises an asparagine residue at position 297. Said sequence may further comprise a glycine residue at position 298 and/or an alanine residue at position 295. Said sequence may further comprise leucine residues at positions 234 and 235.

In a further embodiment the Fc receptor binding polypeptide comprises the CH2 constant domain or a fragment thereof which comprises an asparagine residue at position 297. Said sequence may further comprise a glycine residue at position 298 and/or an alanine residue at position 295. leucine residues at positions 234 and 235.

The positions of specific amino acids residues as herein reference and/or modified refers to residue numbering applied to the constant domains of the human IgG heavy chain. Generally, this numbering therefore relates to the numbering prescribed to the amino acid residues which are present in the CH2 constant domain of a human IgG immunoglobulin. It will be understood that the Fc receptor binding protein of the present invention may comprise a fragment, derivative, analogue or variant of the CH2 constant domain. In such cases, the numbering applied to the amino acid residues which are present in this sequence may vary depending upon the sequence length. Accordingly, for example, the asparagine residue which is present at residue 297 of the defined CH2 domain constant region as derived from the Kabat database may not correlate with the position of that residue at position 297 of the amino acid sequence of the fusion protein or of the Fc receptor binding polypeptide. In such instances, it will be appreciated that the skilled person will use an alignment process to identify where the residue equivalent to the asparagine 297 residue of the Kabat database defined CH2 human IgG is present on the Fc receptor binding polypeptide sequence. The numbering prescribed to particular amino acids in relation to the Fc receptor binding domain, as applied herein, is based on the numbering of the equivalent residue as defined for the IgG heavy chain constant domain from the Kabat database sequence. The same applies mutatis mutandis to other residues of significance to the Fc receptor binding protein, such as the serine residue at position 298 and/or the glutamine residue at position 295, particularly when the polypeptide is derived from, or a fragment of a CH2 constant domain.

In one embodiment the Fc receptor binding protein comprises the amino acid sequence of SEQ ID NO:1. As such, the fusion protein of said embodiment comprises one or more antigenic polypeptide sequences along with an Fc receptor binding polypeptide comprising the amino acid sequence of SEQ ID NO:1.

In a still further embodiment, the Fc receptor binding protein comprises a fragment, variant or derivative of the sequence as shown in SEQ ID NO:1, wherein said fragment, variant or derivative has the biological activity of the polypeptide having SEQ ID NO:1.

SEQ ID NO: 1:
PKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKATPPVLDSDGSFFLYSKLTVDKGR
WQQGNVFSCSVMHEALHNHYTQKSLSLSPGK

In particular, SEQ ID NO:1 details a polypeptide which can bind to an Fc receptor. The sequence can be aligned with residues 216 to 447 of the determined sequence for the heavy chain constant region of IgG as defined on the Kabat Database of Sequences of Proteins of Immunological Interest (www.kabatdatabase.com). On the basis that the first amino acid reside of SEQ ID NO:1 is taken to be numbered as residue 216 and the last residue numbered as residue 447, then an asparagine (Asn, N) residue, which can be glycosylated, is provided at residue 297. The glycine residue (G, Gly) at residue 415 of SEQ ID NO:1 represents a substitution of the serine (S, Ser) residue present in the defined Kabat database sequence. SEQ ID NO:1 further comprises an alanine (A, Ala) residue at position 393 which replaces the threonine residue (Thr, T) provided in the defined Kabat database sequence. The sequence of SEQ ID NO:1 may be further mutated such that the serine (Ser, S) residue at position 298 can be substituted with an alanine residue (Ala, A). Furthermore, in order to create an mutant version of the fusion protein which comprises the sequence of SEQ ID NO:1 as the Fc receptor binding polypeptide, the two leucine residues provided at residues 234 and 235 of SEQ ID NO:1 may be mutated, for example to residues such as valine and alanine respectively, in order to impair Fc receptor binding.

In a further embodiment, the Fc receptor binding polypeptide having the sequence of SEQ ID NO:2 may be mutated such that the alanine (Ala, A) residue at position 393 is changed to a threonine (Thr, T) residue as is present in the Kabat database sequence. The sequence of SEQ ID NO:2 is provided below:

SEQ ID NO: 2:
PKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKGR
WQQGNVFSCSVMHEALHNHYTQKSLSLSPGK

Accordingly in a further embodiment, the Fc receptor binding protein comprises a sequence as shown in SEQ ID NO:2. As such, the fusion protein of said embodiment comprises one or more antigenic polypeptide sequences and an Fc receptor binding polypeptide comprising the amino acid sequence of SEQ ID NO:2.

In a still further embodiment, the Fc receptor binding protein comprises a fragment, variant or derivative of the sequence as shown in SEQ ID NO:2 has the biological activity of the polypeptide having SEQ ID NO:2.

The sequence of SEQ ID NO:2 may be further mutated such that the serine (Ser, S) residue at position 298 is substituted with an alanine residue (Ala, A). Furthermore, in order to create an mutant version of the fusion protein which comprises the sequence of SEQ ID NO:2 as the Fc receptor binding polypeptide, the two leucine residues provided at residues 234 and 235 of SEQ ID NO:2 may be mutated, for example to residues such as valine and alanine respectively, in order to impair Fc receptor binding.

FIG. 5 shows an alignment of SEQ ID NO:1 with residues 216 to 447 of the human IgG heavy chain constant domains, the latter sequence being referred to as SEQ ID NO:3.

In certain further embodiments the Fc receptor binding polypeptide comprises a fragment, analogue or derivative of a Cγ2 domain derived from a human IgG immunoglobulin, typically IgG1, IgG2 or IgG3.

In certain further embodiments, the Fc receptor binding polypeptide in addition to comprising SEQ ID NO:1, SEQ ID NO:2 or the Cγ2 domain derived from a human IgG immunoglobulin may further comprise a further constant domain, for example, but not limited to, the Cγ3 constant domain and/or a hinge region, such as the hinge which comprises the proline rich stretch of amino acids present between the Cγ2 and Cγ3 constant domains, said hinge region conferring structural flexibility on the Fc receptor binding polypeptide.

The one or more antigenic polypeptide sequences may be linked to the Fc receptor binding polypeptide, such as SEQ ID NO:1 or SEQ ID NO:2, by means of a covalent bond. Alternatively a non-covalent bond may be used. In further embodiments, a linker moiety or spacer may be used to conjoin the sequences.

Typically the Fc receptor binding polypeptide is capable of binding to Fc receptor FcγRI (CD64) and/or to Fc receptor FcγRII (CD32), and/or to Fc receptor FcγRIII (CD16). In certain embodiments, the Fc receptor binding polypeptide is capable of binding to an FcR that is coupled to the FcR-gamma chain via a salt-bridge in its transmembrane sequence.

As herein defined, the term “antigenic polypeptide sequence” refers to a polypeptide which induces a long term protective immune response against said polypeptide sequence when it is administered to a subject.

The antigenic polypeptide may be derived from any suitable source, for example a pathogenic organism or a tumour specific antigen, or can be derived from a non-pathogenic disease, such as an autoimmune disease or a neurodegenerative disease. The antigenic peptide may be derived from the identification of an endogenous normal protein which is associated with the pathogenesis of a given condition. In such instances, the whole protein or fragments, analogues or derivatives thereof can be used.

In certain further embodiments, the antigenic polypeptide sequence is selected from the group consisting of a viral polypeptide, a bacterial polypeptide, a tumour specific polypeptide and an immunomodulatory polypeptide. The polypeptide may be a complete protein, a fragment thereof or an analogue or derivative of a protein or polypeptide.

Suitably the antigenic polypeptide is a viral polypeptide, wherein the virus is selected from the group consisting of: HIV-1, HIV-2, Hepatitis-B, Hepatitis-C and Influenza type A. Where the viral polypeptide is derived from avian influenza virus, the polypeptide may be derived from Influenza type A virus selected from the group consisting of: H5N1, H9N2, H7N1, H7N2, H7N3 and H7N7.

In certain further embodiments, site directed mutagenesis may be performed on the polynucleotide sequence encoding the antigenic polypeptide. This approach has the additional advantage of introducing desirable genetic changes such as the removal of protease cleavage sites or variable loops in the antigenic protein. For example the removal of the HA1/2 cleavage site in influenza HA or the loops in HIV gp120 would yield fusion proteins which induce immune responses that would be redirected to the less variable regions of these antigens. In further embodiments, an antigenic polypeptide sequence incorporating the above mutations can be produced entirely by chemical synthesis.

In a further embodiment the antigenic peptide may consist of the product of more than one gene, for the example the production of heterologous fusion antigens such as HIV tat/rev, influenza HA/NA or M. tuberculosis Ag85/ESAT-6 or Rv1025/1196 TB gene fusions. Such heterologous fusion antigens may further be produced by chemical synthesis.

Accordingly in further embodiments the antigenic polypeptide comprises an antigenic polypeptide fragment which comprises a fusion of 2 or more antigenic polypeptides. The 2 or more antigenic polypeptides may be derived from the same pathogen, or from different pathogens. For example, in certain embodiments, the antigenic polypeptide comprises a fusion of the tat and rev polypeptides of HIV or fragments thereof. In a further embodiment the antigenic polypeptide comprises a fusion of haemagglutinin (HA or H) and neuraminidase (NA or N) or fragments thereof as derived from influenza. In a still further embodiment the antigenic polypeptide may comprise a fusion of the polypeptides encoded by the Ag85/ESAT-6 or Rv1025/1196 genes of tuberculosis. In further still embodiments, the heterologous antigenic polypeptides can be produced synthetically, for example by chemical synthesis. Suitable chemical synthesis techniques will be well known to the person skilled in the art, and include, but are not limited to standard liquid or solid-phase peptide synthesis methods.

In further embodiments, the antigenic polypeptide is a T cell epitope. The T cell epitope may be a pathogen epitope or a non-pathogen epitope. In all embodiments, the T cell epitope should be suitable for mediating a cytotoxic T lymphocyte (CTL) response when administered to a subject.

In certain embodiments, the fusion protein further comprises one or more additional heterologous polypeptides, for example immunomodulatory peptides, such as a cytokine.

A further aspect of the present invention provides an immunogenic composition comprising the fusion protein of the first aspect of the invention.

In certain further embodiments, the immunogenic composition further comprises an adjuvant.

In a further aspect, the invention extends to a method of inducing an immune response in a subject comprising the step of administering the immunogenic composition of the foregoing aspect of the invention to a subject in an amount sufficient to induce an immune response. Typically the immune response is a cytotoxic T lymphocyte (CTL) response.

In a further aspect, the present invention provides a polynucleotide sequence encoding the fusion protein of the first aspect of the invention or which encodes at least one polypeptide which is a component of the fusion protein of the first aspect of the present invention.

In one embodiment, the polynucleotide encodes amino acid having the amino acid sequence of SEQ ID NO:1 or SEQ ID NO:2. Said amino acid can thereafter be conjoined to at least one antigenic peptide fragment in order to form the fusion protein of the invention.

A further aspect of the present invention provides a vector which comprises at least one polynucleotide sequence which encodes the fusion protein of the present invention or polypeptide thereof. Suitably the vector comprises at least one control sequence element operably linked to the at least one polynucleotide sequence. Said control elements are typically selected depending upon the cell line in which the vector is to be expressed.

A still further aspect of the invention provides a host cell which comprises the vector or the polynucleotide sequences or the fusion protein of the present invention.

A yet further aspect of the present invention provides a method of producing a fusion protein according to the invention, the method comprising the step of incubating a cell, which comprises a polynucleotide, which encodes a fusion protein comprising one or more antigenic polypeptide sequences and an Fc receptor binding polypeptide which binds to an Fc receptor with a binding affinity sufficient to cause internalisation of the bound Fc receptor, or a vector comprising said polynucleotide, under conditions suitable for producing the fusion protein.

In one embodiment the Fc receptor binding polypeptide comprises the amino acid sequence of SEQ ID NO:1. In a further embodiment the Fc receptor binding polypeptide comprises the amino acid sequence of SEQ ID NO:2.

According to a further aspect of the present invention there is provided a method for inducing an immune response in a subject, the method comprising the steps of:
providing a fusion protein comprising one or more antigenic polypeptide sequences and an Fc receptor binding polypeptide which binds to an Fc receptor with a binding affinity sufficient to cause internalisation of the bound Fc receptor, and
administering a therapeutically effective amount of said fusion protein to a subject in which the induction of the immune response against said antigenic polypeptide or a fragment thereof is desired.

In certain embodiments the method extends to inducing an immune response for the treatment of infection with a pathogenic disease, the method further comprising the steps of:
obtaining an antigenic polypeptide sequence derived from the pathogen which is causative of the disease or a product derived from the pathogen,
using said antigenic polypeptide sequence in the formation of the fusion protein of the first aspect of the invention, and
administering a therapeutically effective or prophylactically effective amount of a composition comprising the fusion protein to a subject in need of such treatment.

Typically the antigenic polypeptide is derived from a bacterial pathogen, a viral pathogen, a parasitic pathogen or a protozoan pathogen.

In further embodiments the method further extends to inducing an immune response for the treatment of a non-pathogenic disease condition, the method further comprising the steps of:
obtaining an antigenic polypeptide sequence specific to the non-pathogenic disease condition,
using said antigenic polypeptide sequence in the formation of the fusion protein, and
administering a therapeutically effective or prophylactically effective amount of a composition comprising the fusion protein to a subject in need of such treatment.

In one embodiment the fusion protein comprises an Fc receptor binding polypeptide comprising the amino acid sequence of SEQ ID NO:1. In a further embodiment the Fc receptor binding polypeptide comprises the Fc receptor binding polypeptide comprising the amino acid sequence of SEQ ID NO:2.

Typically the non-pathogenic antigenic polypeptide is selected from the group consisting of; a tumour specific polypeptide, a polypeptide which is specific to a neurodegenerative disease and a polypeptide which is specific to an autoimmune disease.

Suitably the immune response is a cytotoxic T cell response.

In one embodiment, the subject is a mammal. In further embodiments the mammal is a human.

Typically the fusion protein is administered intravenously, subcutaneously or intramuscularly, these routes being preferred as these tissues contain dendritic cells which express Fc receptors and further said tissues lack high levels of serum IgG which may compete for Fc receptor binding.

According to a further aspect of the present invention there is provided a method for inducing an immune response in a subject, the method comprising the steps of:
administering a therapeutically effective amount of a polynucleotide or a gene delivery vector which encodes a fusion protein according to the first aspect of the invention to cells of a subject under conditions that permit the expression of the polynucleotide and production of the fusion protein, thereby eliciting an immune response to the fusion protein.

Suitably, the immune response is a cytotoxic T cell response.

In one embodiment, the subject is a mammal. In a further embodiment the mammal is a human.

Typically, the gene delivery vector is administered subcutaneously, intravenously or intramuscularly.

In certain further embodiments, wherein the cells of the subject are transfected with the polynucleotide or gene delivery vehicle, the transfection is performed ex-vivo and the transfected cells are then re-introduced into the subject.

In an alternative embodiment, the subject's cells are transfected with the polynucleotide or gene delivery vehicle in-vivo.

A yet further aspect of the present invention provides a pharmaceutical composition for use in inducing an immune response, wherein said pharmaceutical composition comprises a fusion protein comprising one or more antigenic polypeptide sequences and an Fc receptor binding polypeptide which binds to an Fc receptor with a binding affinity sufficient to cause internalisation of the bound Fc receptor, along with a pharmaceutically acceptable excipient, carrier or diluent.

In one embodiment the Fc receptor binding polypeptide comprises the amino acid sequence of SEQ ID NO:1. In a further embodiment the Fc receptor binding polypeptide comprises the amino acid sequence of SEQ ID NO:2.

A further aspect of the present invention provides a fusion protein comprising one or more antigenic polypeptide sequences and an Fc receptor binding polypeptide which binds to an Fc receptor with a binding affinity sufficient to cause internalisation of the bound Fc receptor for use as a medicament.

In one embodiment the Fc receptor binding polypeptide comprises the amino acid sequence of SEQ ID NO:1. In a further embodiment the Fc receptor binding polypeptide comprises the amino acid sequence of SEQ ID NO:2.

A still further aspect of the invention provides for the use of a fusion protein comprising one or more antigenic polypeptide sequences and an Fc receptor binding polypeptide which binds to an Fc receptor with a binding affinity sufficient to cause internalisation of the bound Fc receptor for the preparation of a medicament for the treatment and/or prevention of disease.

In one embodiment the Fc receptor binding polypeptide comprises the amino acid sequence of SEQ ID NO:1. In a further embodiment the Fc receptor binding polypeptide comprises the amino acid sequence of SEQ ID NO:2.

In certain embodiments the disease is a pathogenic disease. In such cases, typically the pathogenic disease is a viral infection, for example HIV-1, HIV-2, Hepatitis-B, Hepatitis-C or Influenza. Where the viral infection is Influenza type A, the subtype may be selected from: H5N1, H9N2, H7N1, H7N2, H7N3 and H7N7.

In further embodiments the disease is a non-pathogenic disease, for example, but not limited to: a cancerous or malignant condition, a neurodegenerative disease and an autoimmune disease.

Typically, the use of the medicament results in the induction of a cytotoxic T lymphocyte (CTL) response.

In one embodiment the medicament further comprises an adjuvant.

In one embodiment, the medicament is provided for intramuscular or intradermal administration.

Typically, the medicament is a vaccine which can be administered prophylactically in order to mediate long term protective immunity in a subject against a pathogen from which the antigenic polypeptide fragment of the fusion protein is derived.

Typically, the medicament is a vaccine which can be administered therapeutically in order to mediate long term protective immunity in a subject against a cancerous condition from which the antigenic polypeptide fragment of the immunoconjugate is derived, said antigenic polypeptide being a tumour-specific antigen.

In certain embodiments, the disease is a viral infection, such as, but not limited to, HIV-1, HIV-2, Hepatitis-B, Hepatitis-C or Influenza.

A still further aspect of the invention provides for the use of a polynucleotide which encodes a fusion protein comprising one or more antigenic polypeptide sequences and an Fc receptor binding polypeptide which binds to an Fc receptor with a binding affinity sufficient to cause internalisation of the bound Fc receptor for the preparation of a medicament for the treatment and/or prevention of disease.

Typically, the use of the medicament results in the induction of a cytotoxic T lymphocyte (CTL) response.

The present inventor has also determined that the fusion protein of the present invention may be administered along with other therapies to prevent and/or treat disease. For example, a composition comprising a fusion protein according to the present invention wherein the antigenic polypeptide is a viral polypeptide derived from an infectious virus, said composition further comprising at least one pharmaceutically acceptable carrier, may be administered in combination with at least one further therapeutic agent which has a prophylactic and/or therapeutic effect on the development of viral infection. This provides a combination therapy which has utility in relation to a viral infection which has a particularly high pathogenicity.

Accordingly, a further aspect of the present invention provides a method for preventing and/or treating a microbial infection in a subject, the method comprising the steps of;
providing a composition comprising a fusion protein comprising one or more antigenic polypeptide sequences and an Fc receptor binding polypeptide which binds to an Fc receptor with a binding affinity sufficient to cause internalisation of the bound Fc receptor,
administering a therapeutically effective amount of said composition to a subject in need of treatment, and
further administering a therapeutically effective amount of a suitable secondary anti-microbial compound.

In one embodiment, the secondary anti-microbial compound is administered along with the fusion protein, however, in further embodiments, the secondary anti-microbial compound may be administered before or after the fusion protein has been administered.

In one embodiment, the antigenic polypeptide is derived from a viral pathogen and the secondary compound is an anti-viral compound. The secondary anti-viral compound may be selected from the group comprising; ribavirin, amantadine, rimantadine, oseltamivir (TAMIFLU™) and zanamivir.

In one embodiment, the polypeptide antigenic polypeptide is derived from a type A influenza virus. In further embodiments, the type A Influenza virus is of the strain H5N1, H9N2, H7N2, H7N3 or H7N7. In yet further embodiments the type A influenza virus may comprise haemagglutinin of subtype H5, H7 or H9 along with any neuraminidase subtype.

In another embodiment, the antigenic polypeptide is derived from a hepatitis virus. In further embodiments, the hepatitis virus is a type B virus and the preferred anti-viral compound is lamuvidine. In a yet further embodiments, the hepatitis virus is a type C virus and the preferred anti-viral compound is selected from ribavirin and Interleukin 2 (IL-2) or combinations thereof.

According to a yet further aspect of the present invention there is provided the use of the fusion protein of the present invention and an anti-viral compound in the preparation of a combined medicament for the treatment or prevention of infection with type A Influenza subtype H5, H7 or H9. In one embodiment the type A Influenza subtype is of the strain H5N1, H9N2, H7N1, H7N2, H7N3 or H7N7. In a preferred embodiment, the secondary anti-viral compound may be selected from the group comprising; ribavirin, amantadine, rimantadine, oseltamivir (TAMIFLU™) or zanamivir.

In another embodiment, the fusion protein of the invention may be administered as a medicament within an interruptive anti-viral therapy schedule in combination with anti-viral compounds.

In yet another embodiment, the polypeptide conjugate of the invention may be administered as a medicament in interruptive anti-viral therapy of HIV/AIDS in combination with triple-therapy anti-viral compounds.

Preferred features of each aspect and embodiment of the invention are as for each of the other aspects mutatis mutandis unless the context demands otherwise.

### BRIEF DESCRIPTION OF THE DRAWINGS

FIG. 1 shows a graph showing weight loss data for animals immunised according to the protocol of Example 3,

FIG. 2 shows the results of FACS analysis showing the binding of fusion proteins to Fc receptors on human THP-1 cells,

FIG. 3 shows the results of FACS analysis showing the binding of fusion proteins to Fc receptors on RAW cells (mouse dendritic cells),

FIG. 4 shows the results of FACS analysis showing the binding of fusion proteins to Fc receptors on human THP-1 cells, and

FIG. 5 shows an alignment showing a comparison of the amino acid sequences of SEQ ID NO:1 (upper sequence denoted *) with residues 216 to 447 (SEQ ID NO:3) of the heavy chain constant domain of IgG as derived from the Kabat database (lower sequence denoted **).

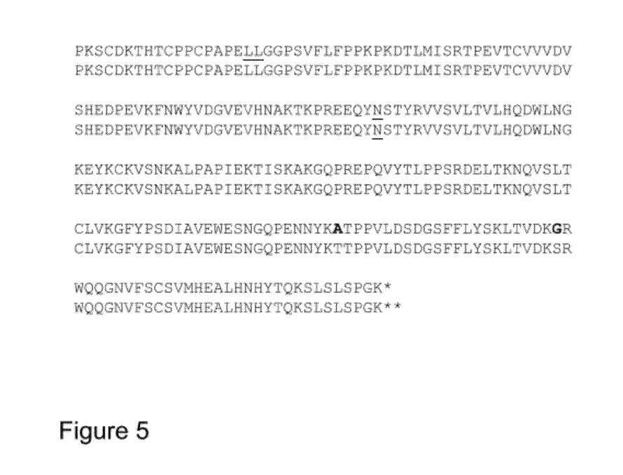

### DETAILED DESCRIPTION OF THE INVENTION

The present invention provides a fusion protein which comprises an Fc receptor binding protein which binds to an Fc receptor with a sufficient binding affinity to cause internalisation of the Fc receptor and the associated bound complex.

Fc receptor internalisation allows efficient antigen presentation to stimulate T cell responses. Fc receptor internalisation is significantly more efficient than pinocytosis for stimulating helper T cell responses. However, the inventor has surprisingly shown that internalisation of Fc receptors along with associated antigens can result in effective antigen processing which can result in not only T-helper cell responses, but also cytotoxic T lymphocyte (CTL) responses. Cytotoxic T lymphocytes (CTLs) target and destroy diseased or abnormal cells by direct cytotoxicity, and by providing specific and non-specific help to other immunocytes, such as macrophages, B cells, and other types of cells. The internalised Fc receptor bound fusion proteins of the invention are shown to elicit a specific CTL response directed to the antigen or antigenic fragment(s) provided within the fusion protein. This therefore provides an effective means for inducing long term protective immune responses where desired against conditions such as infectious diseases, cancerous conditions and neurodegenerative diseases.

The fusion protein of the invention may be defined by the primary amino acid sequence ABC, wherein A relates to one or more antigenic polypeptide sequences, B is an optional linker moiety and C is an Fc receptor binding polypeptide which binds to an Fc receptor with a binding affinity sufficient to cause internalisation of the bound Fc receptor.

In one embodiment A defines the N terminal (amino) portion of the polypeptide and C defines the C-terminal (carboxyl) terminal of the polypeptide. In such an embodiment, if a linker is used to conjoin A and C, the linker would be conjoined to the C-terminal of the amino acid sequence defining the antigenic polypeptide fragment, and to the N-terminal of the amino acid sequence defining the polypeptide which binds to the Fc receptor.

In one embodiment of the present invention, the Fc receptor binding polypeptide of the fusion protein is derived from at least one constant domain of the Fc portion of a human immunoglobulin, typically the CH2 domain or a variant thereof. Constant regions derived from human immunoglobulins are particularly preferred. Different classes of human immunoglobulin may be suitable for providing the Fc receptor binding polypeptide of the immunoconjugate.

The class or ‘isotype’ of an antibody is defined by its heavy chain. In particular, immunoglobulins of the isotype IgG are most preferred, however antibodies of isotypes IgA, IgM, IgE and IgD may also have utility in various further embodiments of the present invention.

Typically, the Fc receptor binding polypeptide of the fusion protein is derived from a human antibody, typically an antibody of the isotype IgG. IgG has a number of subclasses, such as IgG1, IgG2a, IgG2b, and IgG3. Each IgG subclass has constant domains with a very high level of homology, however each isotype differs significantly in the hinge region. Suitably, the Cγ2 constant domain derived from the heavy chain of human IgG1 immunoglobulin is used, or a fragment, analogue or derivative thereof.

However, although any subclass of IgG has utility in the present invention, typically a sequence encoding at least one constant domain or a fragment thereof derived from the IgG subclass IgG3 would be used. The inventor has identified that the structure of the IgG3 antibody is also particularly suited to the present invention, due to the presence of the extended hinge region. The hinge region of an antibody is generally located between the CH1 constant domain and the CH2 constant domain and is thought to provide structural flexibility to the antibody in order to facilitate binding by the Fab portion of the antibody.

The constant domains of an antibody have importance in directing the immune response, and, in particular, the recruitment of effector functions which mediate the immune response following antibody binding. The type of effector functions which are induced following the binding of an antibody can be dependent upon the constant regions of the heavy chain and, in particular, the CH2 and CH3 domain regions. The ability to induce a response from the immune system which provides long term protective immunity is important in the continued protection against a pathogen.

An integral part of such a response is the binding and activation of FcRs. FcRs are present on many cells of the immune system, such as antigen presenting cells, and, in particular, macrophages, B cells, neutrophils, mast cells, NK cells and follicular dendritic cells. Ligand binding to FcRs results in the activation of a number of effector mechanisms such as; complement activation, ADCC (antibody dependent cell mediated cytotoxicity), and the release of immune mediators such as cytokines. Fc receptors are antibody class specific and isotype selective.

The utility of the present invention lies in the ability of the fusion protein of the present invention to bind to FcRs with a sufficiently high binding affinity so as to induce the internalisation of the bound FcR within the cell upon which the FcR is expressed. This binding results in the activation of a number of effector mechanisms, such as the release of immune mediators, such as chemokines and cytokines. As discussed hereinbefore, the binding of the FcR can also result in the internalisation of the FcR in instances where the FcR is bound by a ligand at a sufficiently high binding affinity (binding avidity). Antigen presentation can also be upregulated, with this resulting in enhanced uptake and presentation of the fusion protein by the cell which expressed the Fc receptor.

There are three types of FcR known to have binding specificity to IgG, with each having a specific function ascribed thereto. FcγRI (also known as CD64) is present on macrophages and monocytes. FcγRII (CD32) is expressed on B cells, macrophages, neutrophils, and monocytes. Lastly, FcγRIII (CD16) is expressed by macrophages, NK cells and neutrophils. FcRs specific to other antibody isotypes include; FcεRI and FcεRII, which are expressed by B cells, monocytes and follicular dendritic cells and which have specificity for IgE Fc portions; FcαR, which has specificity for IgA Fc portions, and FcμR which has specificity for IgM Fc domains.

Accordingly, in one embodiment, the Fc receptor binding polypeptide portion of the immunoconjugate can bind to at least one of the Fc receptors selected from the group comprising; FcγRI (CD64), FcγRII (CD32), or FcγRIII (CD16). In a preferred embodiment, the Fc receptor binding polypeptide can bind to both FcγRI (CD64) and FcγRIII (CD16). In a more preferred embodiment, the Fc receptor binding polypeptide can bind to FcRs that are coupled to the FcR gamma chain via a salt-bridge in their transmembrane domain.

Typically, the Fc portion of the immunoconjugate of the present invention is comprised of an amino acid sequence defining the CH2 constant domain of the IgG1 antibody or a variant or fragment thereof. In particular, the CH2 constant domain or variant or fragment thereof (also referred to as the C2 constant domain), may be derived from the heavy chain of the human IgG1 antibody.

Alternatively, the Fc portion of the fusion protein of the present invention is comprised of an amino acid sequence defining the CH2 constant domain of the IgG3 antibody or a variant or fragment thereof. In a preferred embodiment, the CH2 constant domain (also referred to as the C2 constant domain) is derived from the heavy chain of the human IgG3 antibody.

In certain further embodiments the Fc receptor binding polypeptide comprises the amino acid sequence of SEQ ID NO:1. In a yet further embodiment the Fc receptor binding polypeptide comprises the amino acid sequence of SEQ ID NO:2.

The polypeptide encoding the Fc receptor binding polypeptide may be obtained by recombinant methods or alternatively may be produced synthetically. In a further embodiment, the Fc receptor binding domain may be obtained following proteolytic digestion of immunoglobulin molecules, for example by papain digestion of immunoglobulins. The antigenic peptide may be coupled by any method to Fc by any method known in the art including chemical linkages. For example, the conjugation can involve the use of chemical crosslinking molecules, such as the use of heterobifunctional crosslinking agents, such as succinimidyl esters, for example, 3-(2-pyridyldithio)propionate or succinimidyl acetylthioacetate (Molecular Probes Inc. Handbook, Chapter 5, section 5.3).

The antigenic polypeptide fragment may be modified to create derivatives by forming covalent or aggregative conjugates with other chemical moieties, such as glycosyl groups, polyethylene glycol (PEG) groups, lipids, phosphate, acetyl groups and the like.

Covalent derivatives of the polypeptides of the invention can be prepared by linking the chemical moieties to functional groups on the amino acid side chains or at the N-terminus or C-terminus of the antigenic polypeptide.

Suitably, the antigenic polypeptide A is derived from a pathogenic organism. Alternatively, the antigenic polypeptide A is derived from a protein that is responsible for a pathogenic process, said pathogenic process causing illness or disease in a host. The antigenic polypeptide A may be further derived from a pathogen which is causative of an infectious disease. Alternatively, the antigenic polypeptide A is derived from a protein that is responsible for a pathogenic process, said pathogenic process causing illness or disease in a host. Furthermore, the antigenic polypeptide A may be derived from a secreted product or other infectious agent which is derived from a pathogenic organism. The antigenic polypeptide may further be synthetic. Techniques for the production of such synthetic proteins are well known to the person skilled in the art.

In further embodiments, the antigenic polypeptide may be a viral peptide, a bacterial polypeptide, a tumour specific polypeptide, a polypeptide specific to a neurodegenerative disease, a peptide specific to an autoimmune disease or a polypeptide specific to any other non-pathogenic disease.

In further embodiments, the antigenic peptide or fragment thereof is a T cell epitope. T cell epitopes can be predicted using known algorithms or synthesised as peptides and screened using standard T cell assays.

Identified T cell epitopes may be cytotoxic T cell epitopes and may have a preferred amino acid length of from 9 to 20 amino acids.

In a yet further embodiment, the antigenic polypeptide is derived from a secreted product or other infectious agent which is derived from a pathogenic organism. In particular, the secreted product may be derived from a bacterial pathogen and is selected from the group consisting of; leukocidins, streptolysin S, streptolysin O, NADase, hyaluronidase, streptokinases and pyrogenic exotoxins.

In one embodiment, the infectious disease is caused by a microbial pathogen. In a further embodiment, the infectious disease is caused by a bacterial pathogen. In a further embodiment, the infectious disease is caused by a fungal pathogen. In a further embodiment the infectious disease is caused by a viral pathogen. In a further embodiment, the infectious disease is caused by a tumour specific pathogen.

In one embodiment, the antigenic fragment is a viral polypeptide. In a further embodiment, the antigenic polypeptide fragment is derived from a strain of Influenza virus which can cause infection in humans. Typically, the antigenic fragment is haemagglutinin (HA or H) or a fragment thereof derived from an infectious strain of type A influenza virus. Alternatively, the antigenic fragment is the HA3 component of a type A influenza virus.

In another embodiment, the antigenic polypeptide may be derived from type A avian influenza virus. The type A avian influenza virus may be defined by the presence of the haemagglutinin subtype H5, H7 or H9. In further embodiments, the type A Influenza virus is of the strain H5N1, H9N2, H7N1, H7N2, H7N3 or H7N7. In yet further embodiments, the type A influenza virus may comprise haemagglutinin of subtype H5, H7 or H9 along with any neuraminidase subtype.

In another embodiment the infectious disease is hepatitis, in particular that caused by type C hepatitis virus. In yet another embodiment the infectious disease is hepatitis, in particular that caused by type B hepatitis virus. In a yet further embodiment the infectious disease is HIV caused by the HIV-1 or HIV-2 retrovirus.

In further embodiments, the antigenic polypeptide may be derived from any pathogen, at least one antigen from which mediates an immune response following infection of a host by said pathogen. Such a pathogen would in particular be of the group referred to as ‘infectious agents’ or ‘infectious diseases’ and may be a viral infectious disease selected from, but not limited to the group comprising; influenza, rhinovirus (common cold), corona virus (such as severe acute respiratory syndrome (SARS) coronavirus), retroviruses (such as HIV), human paillomavirus, smallpox, rabies, rubella, hepatitis, herpes simplex virus, herpes zoster virus, viral meningitis, yellow fever, west Nile disease, chicken pox (varicella), and food and mouth disease virus (FMDV).

The antigenic polypeptide may be further derived from bacterial infectious diseases selected from, but not limited to the group consisting of; tuberculosis, typhoid, anthrax, bacterial meningitis, cholera, diphtheria, gonorrhoea, legionellosis, leptispirosis, listeriosis, MRSA infection, and pertussis.

The antigenic polypeptide may further be derived from a parasitic infectious disease selected from, but not limited to the group consisting of; leishmaniasis, malaria and trypanosomiasis, fungal infectious diseases such as tinea pedis, and candidiasis, prion infectious diseases such as bovine spongiform encephalopathy and Creutzfeldt-Jakob disease (CJD).

In a yet further embodiment, the antigenic fragment may be derived from a host protein that is responsible for pathogenesis. Such a protein would include, but not be limited to, the amyloid proteins that cause pathogenesis in Alzheimer's disease, the nicotinic acid receptor implicated in nicotine addiction and the cholesterol transferase CTEP implicated in the pathogenesis of atherosclerosis.

The fusion proteins of the present invention have further utility in the treatment of cancerous or malignant conditions. The induction of a cytotoxic T lymphocyte (CTL) response following the administration to a subject of a fusion protein of the invention which comprises a tumour-specific antigen provides a therapy which can significantly inhibit growth of tumour cells when administered in a therapeutically effective amount to a subject in need of such therapy.

The fusion protein, polypeptide encoding the fusion protein, or the vector comprising a polypeptide which encodes the fusion protein may be administered to a subject along with an adjuvant. The adjuvant may be administered simultaneously with, or sequentially to, the administration of the fusion protein.

Adjuvants act to non-specifically stimulate a subject's immune system. Adjuvants which may be provided according to the methods of the present invention include, but are not limited to; alum, MF59, QUIL-A (Quil A saponin), detox, ISCOMs, cytokines, squalene, polyols, polyanions, peptides, proteins, aluminium hydroxide, pluronic, oils and emulsions.

The antigenic polypeptide may be conjoined to the Fc receptor (FcR) binding polypeptide at either its N-(amino) or C-(carboxyl) terminal. Typically however, the amino acid sequence encoding the antigenic polypeptide is provided at the amino (N) terminal region of the immunoconjugate, with the amino acid sequence encoding for the FcR binding polypeptide being provided at the carboxyl terminal of the immunoconjugate.

The first and second sequences, denoted as A and C above, may be joined by any suitable technique, but are typically linked by a covalent bond. However a non-covalent bond may also be used. Alternatively, the polypeptide sequences could be directly conjoined or could be joined by means of a linkage moiety or spacer. A linker moiety such as a hinge region derived from an immunoglobulin may be used. The hinge region serves not only to link the amino acid defining the antigenic polypeptide with the amino acid defining the FcR binding polypeptide of the immunoconjugate, but also provides increased flexibility of the immunoconjugate which can confer improved binding specificity. Typically, the linker acts primarily as a spacer. Typically the linker is comprised of amino acids linked together by peptide bonds. The linker may, for example, comprise from 1 to 20 amino acids. Suitably the linker may comprise amino acid residues which are sterically unhindered, such as glycine and alanine. Suitable forms of linker moieties, are described hereinafter.

The amino acid defining the antigenic fragment of the immunoconjugate may be linked to the linker moiety at either its N-(amino) or C-(carboxyl). Suitable conjugation and linkage techniques would be well known to those skilled in the art and may include, for example, conjugation by thio-ester crosslinking utilising cysteine residues of the Fc polypeptide. Alternatively, the conjugation can involve the use of chemical crosslinking molecules, such as the use of heterobifunctional crosslinking agents, such as succinimidyl esters, for example, 3-(2-pyridyldithio)propionate or succinimidyl acetylthioacetate (Molecular Probes Inc. Handbook, Chapter 5, section 5.3).

Further techniques which may have utility in the conjugation of the antigenic fragment to the Fc binding polypeptide would include the techniques described in published International Patent Applications No WO 94/04690 and WO 96/27011.

Conjugation may further be achieved by genetic means through the use of recombinant DNA techniques that are well know in the art, such as those set forth in the teachings of Sambrook et al. Molecular Cloning: A Laboratory Manual, 2 ed. Vol. 1, pp. 1.101-104, Cold Spring Harbor Laboratory Press, (1989) and F. M. Ausubel et al. Current Protocols in Molecular Biology, Eds. J. Wiley Press (2006), the relevant portions of which are incorporated herein by reference.

In one embodiment, the fusion protein is produced by the use of a contiguous gene fusion of the antigenic domain and the Fc binding polypeptide in a suitable expression vector. In certain embodiments, the gene may comprise a polynucleotide sequence which encodes for a polypeptide comprising the amino acid sequence of SEQ ID NO:1. In further embodiments, the gene may encode for a polypeptide sequence comprising the amino acid sequence of SEQ ID NO:2.

It has been established that glycosylation of the Fc receptor binding polypeptide is important in order to facilitate Fc receptor binding with a sufficient binding affinity to induce Fc receptor internalisation.

In particular, as mentioned hereinbefore, where the Fc receptor binding polypeptide is derived from human IgG1, it is preferable that there is N-linked glycosylation of the asparagine (ASN, N) residue at position 297 of the Cγ2 constant domain (as defined with reference to the sequence of the heavy chain constant region of IgG defined in the Kabat database) as glycosylation of this residue is known to be important for mediating high affinity binding and activation of Fc receptors as detailed hereinbefore.

As such, further aspects of the invention extend to producing the Fc receptor binding polypeptide in a cell type which allows the polypeptide to undergo post-translational modification such that it is glycosylated. In certain embodiments, the Fc receptor binding protein may thereafter be conjoined to a linker moiety and/or the at least one antigenic polypeptide fragment or, alternatively, the fusion protein may be expressed in its entirety in the glycosylating cell.

Accordingly, a yet further aspect of the present invention provides a method for producing a fusion protein, the method comprising the steps of:
expressing an Fc receptor binding polypeptide which binds to an Fc receptor with a binding affinity sufficient to cause internalisation of the bound Fc receptor within a cell which is capable of glycosylating the expressed polypeptides, and
further expressing at least one antigenic polypeptide or a fragment thereof within said cell.

In one embodiment the Fc receptor binding polypeptide comprises the amino acid sequence of SEQ ID NO:1. In a further embodiment the Fc receptor binding polypeptide comprises the amino acid sequence of SEQ ID NO:2.

In one embodiment, the Fc receptor binding protein and the antigenic fragment are expressed from a continuous polynucleotide sequence (nucleic acid sequence) which may be described as a fusion gene. The fusion gene may be formed from removing the stop codon from the polynucleotide sequence encoding the Fc receptor binding protein and conjoining the polynucleotide sequence encoding the antigenic polypeptide or fragment thereof thereto in frame. The polynucleotide may be provided in a vector, wherein expression of the polynucleotide sequence is operably linked to a control element which is compatible with expression in the cell.

The method may further comprise the step of expressing a polynucleotide within the cell which defines a linker moiety or spacer, wherein said linker moiety or spacer joins the Fc receptor binding polypeptide and the antigenic polypeptide.

Although the fusion protein of the invention is provided in monomeric form, in further embodiments, the fusion protein may be provided as a dimeric fusion molecule, this resulting from dimerisation of two fusion proteins. The resulting dimer may be a homodimer comprised of 2 fusion proteins having identical antigenic polypeptides. Alternatively, the dimer may be a heterodimer formed from the joining of 2 fusion proteins having different antigenic polypeptides. Where different antigenic polypeptides are exhibited by the fusion proteins, these antigenic polypeptides may be derived from different pathogenic organisms, or may be derived from different target sites of the same pathogenic organism or pathogenic product. Alternatively, the antigenic polypeptides may be derived from sources other than pathogenic organisms, such as non-pathogenic disease conditions and cancerous conditions as described herein.

In further embodiments of the invention, the fusion proteins may be provided as multimeric molecules. Such multivalent fusion proteins may be formed using Fc binding regions which are derived from Fc regions, or portions thereof of antibodies which are usually present in a multivalent form, for example antibodies of the class IgM (pentameric structure) or IgA (dimeric structure). Where multimeric fusion protein molecules are formed, the fusion proteins may comprise similar or different antigenic polypeptides.

In further still embodiments, the fusion proteins may have conjugated thereto further molecules or compounds which may have utility in mediating or enhancing an immune response.

Methods and techniques to monitor whether an Fc receptor is internalised following binding by its ligand are well known to the person skilled in the art. For example, visualisation of the internalisation of the Fc receptor/fusion protein bound complex may be achieved through the use of techniques such as immunofluorescent or immunocytochemical labelling of cells using techniques that are well known to persons skilled in the art. In one embodiment of the invention a fluorescent protein domain is fused to the Fc binding polypeptide to visualise binding and identify appropriate Fc receptor binding peptides. Such internalisation screens can be used to identify Fc receptor binding proteins which bind to the Fc receptor with sufficient affinity to cause internalisation of the Fc receptor/Fc receptor binding polypeptide complex. Although any suitable antigen presenting cell may be used to perform such studies, dendritic cells are preferably used.

In one embodiment of the invention a fluorescent protein domain is fused to Fc domain sequences to visualise binding and identify appropriate FcR binding fragments with utility for the polypeptide immunoconjugates.

### Administration

The fusion protein of the present invention may be administered alone but will preferably be administered as a pharmaceutical composition, which will generally comprise a suitable pharmaceutically acceptable excipient, diluent or carrier selected depending on the intended route of administration.

Examples of suitable pharmaceutical carriers include; water, glycerol, ethanol and the like.

The fusion protein of the present invention may be administered to a patient in need of treatment via any suitable route. As detailed herein, it is preferred that the composition is administered parenterally by means of the subcutaneous, intradermal or intramuscular route in order to enhance the development of a cytotoxic T lymphocyte (CTL) response. Routes of administration may further include mucosal (including pulmonary) and oral.

In preferred embodiments, the composition is deliverable as an injectable composition. For intravenous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or, Lactated Ringer's injection. Preservatives, stabilisers, buffers, antioxidants and/or other additives may be included, as required.

The effective amount of the composition for the treatment or prevention of disease may be provided in a single dosage regimen or a multi-dose regimen.

The composition may also be administered via microspheres, liposomes, other microparticulate delivery systems or sustained release formulations placed in certain tissues including blood.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition ISBN 0-912734-04-3 and Pharmaceutical Dosage Forms and Drug Delivery Systems; Ansel, H. C. et al. 7th Edition ISBN 0-683305-72-7, the entire disclosures of which is herein incorporated by reference.

### Dose and Dosage Regimen

The composition is preferably administered to an individual in a “therapeutically effective amount”, this being sufficient to show benefit to the individual to whom the composition is administered. The actual dose administered, and rate and time-course of administration, will depend on, and can be determined with due reference to, the nature and severity of the condition which is being treated, as well as factors such as the age, sex and weight of the patient to be treated and the route of administration. Further due consideration should be given to the properties of the composition, for example, its binding activity and in-vivo plasma life, the concentration of the fusion protein in the formulation, as well as the route, site and rate of delivery.

Dosage regimens can include a single administration of the composition of the invention, or multiple administrative doses of the composition. The compositions can further be administered sequentially or separately with other therapeutics and medicaments which are used for the treatment of the condition for which the fusion protein of the present invention is being administered to treat.

Suitably a dosage range of from about 10 ng/kg/day through to 1 mg/kg/day may be utilised when administering the fusion protein of the present invention.

The fusion protein of the present invention is preferably administered to an individual in a “therapeutically effective amount”, this being sufficient to show benefit to the individual. In the case of infectious disease, benefit would include reduction of infection or disease symptoms. In the case of other diseases, benefit would include reduction of disease symptoms.

The actual amount administered, and rate and time-course of administration, will depend on the nature and severity of what is being treated. Prescription of treatment, e.g. decisions on dosage etc, is ultimately within the responsibility and at the discretion of general practitioners and other medical doctors, and typically takes account of the disorder to be treated, the condition of the individual patient, the site of delivery, the method of administration and other factors known to practitioners.

An example of a fusion protein is a protein created by recombinant means from 2 or more proteins. Generally, this is achieved by creating a fusion gene, removing the stop codon from the DNA sequence encoding the first protein and appending the DNA sequence of the second protein in frame. Further proteins can be added where required. The DNA sequence will then be expressed by the cell as a single protein.

In further embodiments, the polypeptides of the fusion protein can be expressed independently of each other, and then joined to form a fusion protein. For example, the antigenic polypeptide component of the fusion protein may be derived directly from a pathogenic organism, a cancerous cell or the like, with this polypeptide being joined to the Fc receptor binding polypeptide of the fusion protein. In further embodiments, the antigenic polypeptide fragment is a fragment of a larger polypeptide derived from a pathogenic organism or cancerous cell, or furthermore may be isolated from a heat shock protein, wherein the antigenic peptide fragment has been bound to the heat shock protein to form a heat shock protein-antigenic peptide fragment complex.

Where the fusion protein of the present invention or the peptides which contribute thereto are produced within a cell, by recombinant means, or otherwise, the peptide or fusion protein is preferably provided in an isolated and purified form. Said isolated and purified form typically comprises the fusion protein or peptide being free or substantially free from material with which it may be associated in the cell in which it is produced.

In order to facilitate purification of the fusion protein when prepared by recombinant means, the fusion protein may further contain ligand binding sequences such as a His-tag, FLAG-Tag or GST-tag which may be joined by means of a linker which includes a cleavage site for proteases or chemical agents which enable liberation of the 2 separate protein entities.

### Production of the Fusion Protein of the Present Invention

Expression, isolation and purification of the polypeptides the invention may be accomplished by any suitable technique, including but not limited to the following:

Expression vectors comprising DNA may be used to prepare the fusion protein or at least one peptide component of the fusion protein of the present invention. A method for producing the fusion proteins or peptides comprises culturing host cells transformed with a recombinant expression vector encoding the fusion protein or peptide components of the present invention under conditions that promote expression of the peptides, then recovering the expressed peptides from the culture. The skilled man will recognise that the procedure for purifying the expressed polypeptides will vary according to such factors as the type of host cells employed and whether the peptide is intracellular, membrane-bound or a soluble form that is secreted from the host cell.

Any suitable expression system may be employed. The vectors include a DNA (polynucleotide sequence) encoding the fusion protein or a peptide of the invention, operably linked to suitable transcriptional or translational regulatory nucleotide sequences, such as those derived from a mammalian, avian, microbial, viral or insect gene. Examples of regulatory sequences include transcriptional promoters, operators, or enhancers, an mRNA ribosomal binding site, and appropriate sequences that control transcription and translation initiation and termination. Nucleotide sequences are operably linked when the regulatory sequence functionally relates to the DNA sequence. Thus, a promoter nucleotide sequence is operably linked to a DNA sequence if the promoter nucleotide sequence controls the transcription of the DNA sequence. An origin of replication that confers the ability to replicate in the desired (E. coli) host cells, and a selection gene by which transformants are identified, are generally incorporated into the expression vector.

In addition, a sequence encoding an appropriate signal peptide (native or heterologous) can be incorporated into expression vectors. A DNA sequence for a signal peptide (secretory leader) may be fused in frame to the nucleic acid sequence of the invention so that the DNA is initially transcribed, and the mRNA translated, into a fusion protein comprising the signal peptide. A signal peptide that is functional in the intended host cells promotes extracellular secretion of the peptide. The signal peptide is cleaved from the peptide during translation, but allows secretion of the fusion protein or peptide from the cell.

Suitable host cells for expression of the peptides of the invention include; prokaryotes, higher eukaryotic cells and yeast. Prokaryotic cells, mammalian cells, and in particular CHO cells, HeLa cells and COS cells are particularly preferred for use as host cells.

Appropriate cloning and expression vectors for use with mammalian, yeast, fungal, prokaryotic and insect cellular hosts are described, for example, in Pouwels et al. Cloning Vectors: A Lab. Manual, Elsevier, N.Y., (1986) (ISBN 0444904018) and Current Protocols in Molecular Biology, Eds. F. M. Ausubel et. al., J. Wiley Press (2006), the disclosures of which are incorporated herein by reference.

Accordingly, the fusion protein of the present invention can be obtained by transforming a vector comprising a polynucleotide sequence encoding the fusion protein into a host cell, culturing the host cell such that the polypeptide encoded by the polynucleotide is expressed and recovering the polypeptide from the host cell, or where a secretion signal is appended to the protein, the surrounding medium. Further details on suitable host cells which may be used for the production of the fusion protein of the invention is provided below.

Prokaryotes include gram-negative or gram-positive organisms. Suitable prokaryotic host cells for transformation include, for example, E. coli, B. subtilis, S. typhimurium and various other species within the genera Pseudomonas, Streptomyces and Staphylococcus. In a prokaryotic host cell, such as E. coli, a polypeptide may include an N-terminal methionine (met) residue to facilitate expression of the recombinant polypeptide in the prokaryotic host cell. The N-terminal Met may be cleaved from the expressed recombinant polypeptide.

Expression vectors for use in prokaryotic host cells generally comprise one or more phenotypic selectable marker genes. A phenotypic selectable marker gene is, for example, a gene encoding a protein that confers antibiotic resistance or that supplies an autotrophic requirement.

DNA encoding the polypeptide immunoconjugate of the present invention may be cloned in-frame into the multiple cloning site of an ordinary bacterial expression vector. Ideally the vector would contain an inducible promoter upstream of the cloning site, such that addition of an inducer leads to high-level production of the recombinant protein at a desired time.

For expression of the recombinant protein, the bacterial cells are propagated in growth medium until reaching a pre-determined optical density. Expression of the recombinant protein is then induced.

Purification and refolding may then be performed using techniques which will be well known to the person skilled in the art.

Mammalian cell or insect host cell culture systems may also be employed to express recombinant polypeptides. These systems have the advantage that the produced polypeptide will have undergone post-translational modifications, such as glycosylation, and this may result in a greater bio-stability of the protein when administered.

Baculovirus systems for production of heterologous proteins in insect cells are well known those skilled in the art. Further, established cell lines of mammalian origin are also known, such as the COS-7 line of monkey kidney cells and Chinese hamster ovary (CHO) cells.

Established methods for introducing DNA into mammalian cells have been described. Transcriptional and translational control sequences for mammalian host cell expression vectors can be excised from viral genomes. Additional control sequences shown to improve expression of heterologous genes from mammalian expression vectors may also be used.

The fusion protein of the present invention may further be expressed in yeast host cells, preferably from the Saccharomyces genus (e.g., S. cerevisiae), Hansuela or Pichia, or in other fungal expression systems such as Aspergillus. Yeast transformation protocols are known to those of skill in the art. One such protocol is described by Hinnen et al., Proc. Natl. Acad. Sci. USA 75:1929, 1978.

With respect to any type of host cell, as is known to the skilled artisan, procedures for purifying a recombinant polypeptide or fragment will vary according to such factors as the type of host cells employed and whether or not the recombinant polypeptide or fragment is secreted into the culture medium.

Two or more polynucleotide sequences can be compared by determining their “percentage identity”. Likewise, two or more polypeptide sequences can be compared by determining their percentage identity. The percent identity of two amino acid sequences or of two nucleic acid sequences may be determined by aligning the sequences for optimal comparison purposes (e.g. gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The invention further extends to fusion proteins according to the invention wherein the Fc receptor binding protein has an amino acid sequence which is at least 90% identical, at least 95% identical, at least 96% identical, at least 97% identical, or at least 98% identical, at least 99% identical, at least 99.5% identical, or at least 99.9% identical to the amino acid sequence of SEQ ID NO:1 or 2. Typically said homologous sequences exhibit the biological function of polypeptides having the amino acid sequence of SEQ ID NO:1 or 2.

The determination of percent identity between two sequences can be accomplished using a mathematical algorithm known to those skilled in the art. The NBLAST and XBLAST programs are examples of computer programs which perform such algorithms. BLAST nucleotide searches can be performed with the NBLAST program to obtain nucleotide sequences homologous to nucleic acid molecules of the invention. BLAST protein searches can be performed with the XBLAST program to obtain amino acid sequences homologous to protein molecules of the invention.

To obtain gapped alignments for comparison purposes, Gapped BLAST can be utilised. Alternatively, PSI-Blast can be used to perform an iterated search which detects distant relationships between molecules (Idem.). When utilising BLAST, Gapped BLAST, and PSI-Blast programs, the default parameters of the respective programs (e.g., XBLAST and NBLAST) can be used. See URL http://www.ncbi.nlm.nih.gov. Further suitable computer based algorithm programs can be utilised and will be known to the person skilled in the art.

Due to the known degeneracy of the genetic code, wherein more than one codon can encode the same amino acid, a polynucleotide sequence which encodes a fusion protein or component polypeptide of the invention can vary in its sequence and still encode a polypeptide having the same amino acid sequence as that encoded from the unaltered polynucleotide sequence. Such variant DNA sequences can result from silent mutations (e.g., occurring during PCR amplification) or can be the product of deliberate mutagenesis of a native sequence.

In another embodiment, the nucleic acid molecules of the invention also comprise nucleotide sequences that are at least 80% identical to a native sequence which encodes a fusion protein of the invention or a polypeptide component thereof such as an Fc receptor binding polypeptide comprising an amino acid binding sequence of SEQ ID NO:1 or 2. Also contemplated are embodiments in which a nucleic acid molecule comprises a sequence that is at least 90% identical, at least 95% identical, at least 98% identical, at least 99% identical or at least 99.9% identical to the fusion protein or component polypeptides.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like.

The nomenclature used to describe the polypeptide constituents of the fusion protein of the present invention follows the conventional practice wherein the amino group (N) is presented to the left and the carboxy group to the right of each amino acid residue.

The expression “amino acid” as used herein is intended to include both natural and synthetic amino acids, and both D and L amino acids. A synthetic amino acid also encompasses chemically modified amino acids, including, but not limited to salts, and amino acid derivatives such as amides. Amino acids present within the polypeptides of the present invention can be modified by methylation, amidation, acetylation or substitution with other chemical groups which can change the circulating half life without adversely affecting their biological activity.

The terms “peptide”, “polypeptide” and “protein” are used herein interchangeably to describe a series of at least two amino acids covalently linked by peptide bonds or modified peptide bonds such as isosteres. No limitation is placed on the maximum number of amino acids which may comprise a peptide or protein. The terms “oligomer” and “oligopeptide” are also intended to mean a peptide as described herein. Furthermore, the term polypeptide extends to fragments, analogues and derivatives of a peptide, wherein said fragment, analogue or derivative retains the same biological functional activity as the peptide from which the fragment, derivative or analogue is derived.

Furthermore the term “fusion protein” as used herein can also be taken to mean a fusion polypeptide, fusion peptide or the like, or may also be referred to as an immunoconjugate. The term “fusion protein” refers to a molecule in which two or more subunit molecules, typically polypeptides, are covalently or non-covalently linked.

As used herein, the term “therapeutically effective amount” means the amount of a fusion protein of the invention which is required to reduce the severity of and/or ameliorate a pathogenic disease, a cancerous condition or a disease such as an autoimmune disease or a neurodegenerative disease or at least one symptom thereof, or which serves to prevent the progression of a pathogenic disease, a cancerous condition or a disease such as an autoimmune disease or a neurodegenerative disease or one or more of the symptoms associated therewith.

As used herein, the term “prophylactically effective amount” relates to the amount of a composition which is required to prevent the initial onset, progression or recurrence of a pathogenic disease, a cancerous condition or a disease such as an autoimmune disease or a neurodegenerative disease or at least one symptom thereof in a subject following the administration of the compounds of the present invention.

As used herein, the term “treatment” and associated terms such as “treat” and “treating” means the reduction of the progression, severity and/or duration of a pathogenic disease, a cancerous condition or a disease such as an autoimmune disease or a neurodegenerative disease or the amelioration of at least one of the symptoms thereof, wherein said reduction or amelioration results from the administration of the fusion protein of the present invention. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition.

As used herein, the term “subject” refers to an animal, preferably a mammal and in particular a human. In a particular embodiment, the subject is a mammal, in particular a human, who has been, or who is going to be exposed to radiation, for example radiation therapy such as chemotherapy or radiotherapy. The term “subject” is interchangeable with the term “patient” as used herein.

A fragment of the fusion protein or of a polypeptide as defined herein generally means a sequence of at least 5 to 7 contiguous amino acids, often at least about 7 to 9 contiguous amino acids, typically at least about 9 to 13 contiguous amino acids, more preferably at least about 20 to 30 or more contiguous amino acids and most preferably at least about 30 to 40 or more consecutive amino acids.

A “derivative” or “variant” of a polypeptide as defined herein, such as those of SEQ ID NO:1, SEQ ID NO:3 or those defining the fusion protein of the invention means a polypeptide modified by varying the amino acid sequence of the polypeptide, for example by manipulation of the nucleic acid encoding the polypeptide or by altering the polypeptide itself. Such derivatives of the natural amino acid sequence may involve insertion, addition, deletion and/or substitution of one or more amino acids, preferably while providing a peptide having interferon alpha binding activity. Typically such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

As is well understood, homology at the amino acid level is generally in terms of amino acid similarity or identity. Similarity allows for ‘conservative variation’, such as substitution of one hydrophobic residue such as isoleucine, valine, leucine or methionine for another, or the substitution of one polar residue for another, such as lysine, glutamic acid for aspartic acid, or glutamine for asparagine. Non-peptide mimetics are further provided within the scope of the invention. Accordingly, the variant has an amino acid sequence that preferably is at least 80% identical to the unaltered polypeptide sequence, most preferably at least 90% identical. The percent identity may be determined, for example, by comparing sequence information using the GAP computer program, version 6.0 described by Devereux et al. (Nucl. Acids Res. 12:387, 1984) and available from the University of Wisconsin Genetics Computer Group (UWGCG).

In one embodiment of the invention the derivative of the Fc receptor binding polypeptide of the fusion protein is sufficient to bind to an FcR and induce its internalisation. If required for purification of the polypeptide, the fusion protein may further contain the protein A and protein G binding regions of the Fc domains or other ligand binding sequences such as a His-tag or GST-tag.

The present invention will now be described with reference to the following examples which are provided for the purpose of illustration and are not intended to be construed as being limiting on the present invention.

### EXAMPLES

### Example 1

### Production of Immunoconjugates

For the production of immunoconjugates (fusion proteins) containing influenza virus haemagglutinin (HA) as the antigenic fragment, HA genes were amplified from viral standards obtained from the National Institute of Biological Standards and Control, Potters Bar (NIBSC) using PCR and cloned into suitable expression vectors containing the Fc gene fragments as fusion proteins. The external domain of the HA3 gene was amplified from A/Bangkok/1/79 (BK79) using the forward 5′-GCGCGGCCATTATGGCCCAAAACCTTCCCGGAAATG-3′ and reverse 5′-GCGCGGCCGAGGCGGCCCCAGTCTTTGTATCCTGAC-3′ primers and the amplified fragment purified, cut with Sfi (sites underlined) and cloned into the Fc fusion protein vector pAc3cFcHis which contains the human IgG1 Fc domain, after amplification in the puc-based vector pAcVSV_{TM}Sfi (SD Chapple and IM Jones J. Biotech. (2002) vol. 95, p 269-27 and Yao Y Y. J Infect Disease (2004) vol. 190, p 91-98). The immunoconjugate thus contained amino acids 17-530 inclusive of the BK79 HA3 and the C2/3 domains of human IgG1 and was rechecked by sequencing of the final plasmid.

The fusion protein was expressed in insect cells by construction of recombinant baculoviruses using the rapid recombination method of co-transfection of plasmid with linearised baculovirus AcMNPV DNA (Yao Y Y J. Infect. Disease (2004) vol. 190, p 91-98). Recombinant baculoviruses were titrated on Sf9 cells in multi-well plates and bulk preparations were done in static cultures using serum free Insect Express media (Invitrogen).

The immunoconjugates (fusion proteins) from these static cultures were also used to check protein expression and FcR binding and internalisation using immunohistochemical staining with HA antisera obtained from NIBSC. Alternatively the commercial BAC-TO-BAC system (Invitrogen) was also used. Immunoconjugates were expressed in HIGHFIVE™ or Sf9 cells using 5L Wave Bioreactor vessels (www.wavebiotech.com) and purified for the preparation of vaccines for efficacy testing by using standard affinity chromatography methods and columns (GE Healthcare, UK).

Similarly, immunoconjugates containing the HA from the H5 avian influenza virus A/Vietnam/1194/04 were made by PCR amplification of the external domain using the following forward 5′-GCGCGGCCATTATGGCCAAGATCAGATTTGCATTGG-3′ and reverse 5′-GCGCGGCCGAGGCGGCCTTGGTAAATTCCTATTG-3′ primers and cloned into the FcR vectors for bulk preparation by baculovirus expression in Sf9 cells described as above. The immunoconjugate contained amino acids 17-530 inclusive of the H5 HA and the C2/3 domains of human IgG1 and was rechecked by sequencing of the final plasmid.

Immunoconjugates were also made using synthetic gene constructs. This approach had the additional advantage of introducing desirable genetic changes such as the removal of protease cleavage sites or variable loops in the influenza HA or HIV gp120 genes or the production of heterologous fusion antigens such as Ag85/ESAT-6 or Rv1025/1196 TB gene fusions. In addition synthetic genes were used to produce FcR binding mutants by sequence changes in the Fc domain such as the replacement of the two leucine residues at positions 234 and 235 by a valine residue and an alanine residue respectively.

### Example 2

### Binding and Internalisation Screens

Immunoconjugates were further selected by their ability to bind to, and induce the internalisation of FcRs. Dendritic cells (DCs) expressing CD32 and CD64 were isolated from peripheral blood using commercial blood DC-purification kits (Miltenyi Biotec). Immunoconjugates were incubated for 15-30 mins at room temperature with DCs in RPMI supplemented with 2% foetal calf serum. Cells were fixed in 0.1% glutaraldehyde and permeabilised using 0.3-0.5% non-ionic detergent such as Triton X100 or Nonidet P40 and the immunoconjugate visualised by immunofluorescence using rabbit antisera against the appropriate HA molecule followed by FITC-labelled goat anti-rabbit second layer. Binding was assessed by flow cytometry (FACS analysis) using THP-1 cells to look at binding to human Fc receptors and RAW cells (mouse dendritic cells) to confirm binding to mouse FcR and ensure validity of animal model for immunogenicity studies.

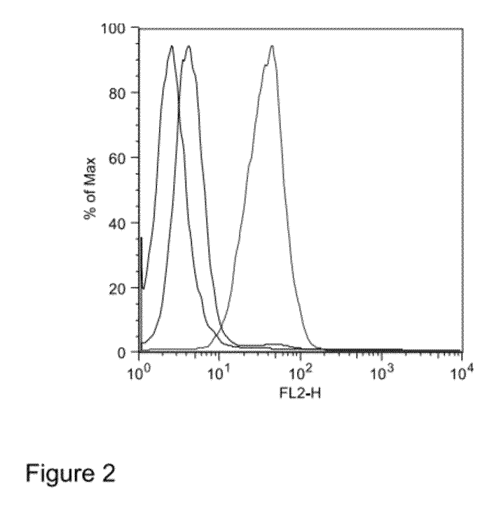

Internalisation was assessed by the observation of intracellular vesicles in a Nikon confocal microscope. An illustration of the data obtained is shown in FIGS. 2, 3, and 4 which demonstrates the ability to distinguish between the two Fc variant immunoconjugates produced as detailed in Example 1 using both RAW and THP-1 cells with the LL variant showing FcR binding while the VA variant did not bind to the FcR or cause internalisation of the FcR.

Specifically, FIG. 2 shows a FACS trace illustration the binding of Fc receptors on human THP-1 by fusion proteins. The centre (middle) peak relates to a fusion protein comprising haemagglutinin derived from Influenza virus as the antigenic peptide, as described in Example 1, and an Fc receptor binding domain wherein the amino acid sequence of the Fc binding polypeptide domain (which, when unaltered comprised the amino acid sequence of SEQ ID NO:1) was mutated to form a variant (the “LL” variant) wherein the two leucine residues present at positions 234 and 235 were replaced by valine and alanine. The peak illustrates that this variant fusion protein does not bind to the Fc receptors present on THP-1 cells.

The furthest right of the 3 peaks shown on FIG. 2 shows the binding of a fusion protein according to the invention which comprises an haemagglutinin antigenic polypeptide and an Fc receptor binding polypeptide which comprises the amino acid sequence of SEQ ID NO:1. The position of this peak illustrates binding of this fusion protein to Fc receptors present on the THP-1 cells.

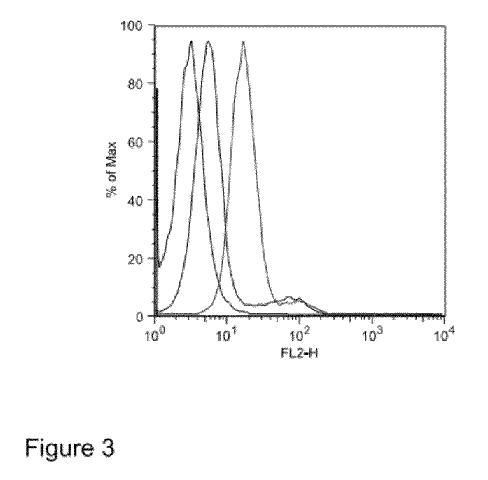

FIG. 3 shown the binding of the above described 2 fusion proteins to Fc receptors present on RAW cells. Binding by the variant fusion protein is illustrated by the middle peak, this showing that there is no binding of the variant fusion protein (with the leucine substitutions present within the sequence of the Fc receptor binding polypeptide). The binding mediated by the un-mutated fusion protein is illustrated by the right hand peak, this confirming that the fusion protein does bind to Fc receptors present on murine RAW cells.

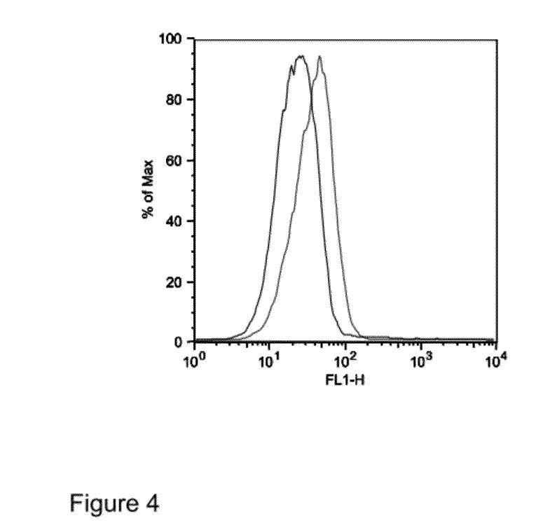

FIG. 4 shows the binding of an alternative fusion protein to Fc receptors present on human THP-1 cells. The left hand peak of FIG. 4 relates to the binding of a variant fusion protein comprising an antigenic polypeptide derived from gp120 of HIV, and further wherein the Fc receptor binding polypeptide comprises the amino acid sequence of SEQ ID NO:1 which has been subjected to the double leucine mutation as described above. The position of the peak shows that binding to Fc receptors by this variant does not occur. The right hand peak relates to a fusion protein comprising an antigenic polypeptide derived from gp120 of HIV and an Fc receptor binding polypeptide which comprises the amino acid sequence of SEQ ID NO:1. The position of the peak illustrates that this fusion protein binds to Fc receptors present in the THP-1 cells.

### Example 3

### Protection Against Heterologous Virus (Drift of Viral Sequence by Genetic Mutation)

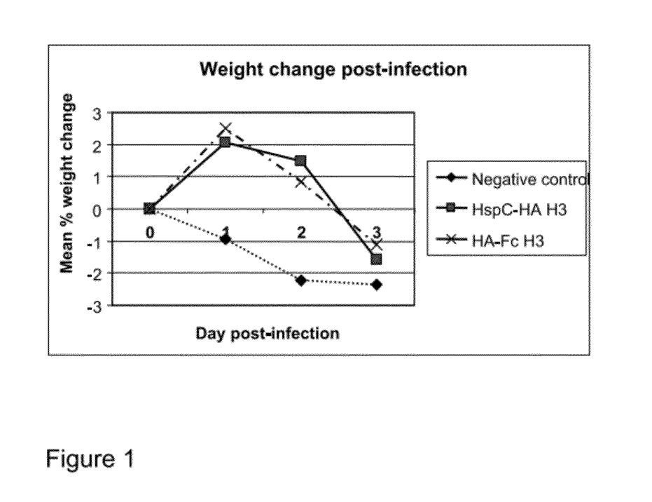

Immunoconjugates of HA3 derived from the A/Bangkok/1/79 virus coupled to the Fc domain from IgG1 were used to immunize balbc mice which were then challenged with a heterologous virus A/Victoria/75 (H3N2) which contains strain mutations that result in 3 drift events separation from the vaccine strain. The haemagglutinin inhibition (HAI) titres of the antisera induced in the immunized animals were assayed using both chicken and turkey erythrocytes. The ability of the immunoconjugate vaccine to both prevent weight loss and reduce viral load in the lungs was used to assess the level of protection in the immunized animals. Animals were vaccinated at day 0, 13 and 27 with 5 ug of immunoconjugate without any adjuvant and challenged at day 41 with a non-lethal dose of infectious heterologous virus. The immunised animals showed both a marked reduction in weight loss (FIG. 1) and a 3-fold reduction in lung viral titres showing that the immunoconjugate vaccines protected the animals against viral infection with a heterologous virus. Immunoconjugates containing the VA variant showed no protection, that is no reduction in lung viral titre was observed.

However, the immunised animals showed no detectable HAI titres either before or after challenge, even if turkey erythrocytes were used to improve HAI sensitivity. These results were especially surprising as HAI titres are widely regarded as indicative of protection and the induction of HAI titres is in fact one of the main criteria used to licence the annual influenza vaccine. It thus appears that the ability of the Fc-immunoconjugates to induce a cellular immune response is sufficient to protect against influenza even in the absence of a strong humoral immune response. The immunity elicited by the immunoconjugate of this invention is even more surprising as it gives protection not just against heterologous strains that are three drift events apart but it does so at a third of the usual dose given to elicit protection against homologous strains in the annual influenza vaccine.

### Example 4

### Protection Against a Pandemic Virus (Avian Influenza)

Immunoconjugates of HA5 derived from the A/Vietnam/1194/2004 (H5N1) avian influenza virus coupled to the Fc domain from IgG1 were used to immunize balbc mice which were then challenged with a homologous virus containing an assortment of the A/Vietnam/1194/2004 HA5 with a PR8 virus (NIBRG-14).

Previous work in this mouse model has shown that HA5 subunit vaccines do not protect against infection unless used at high doses in the presence of adjuvants. The haemagglutinin inhibition (HAI) titres of the antisera induced in the immunised animals were assayed using chicken erythrocytes. The ability of the immunoconjugate vaccine to prevent both weight loss and reduce viral load in the lungs was used to assess the level of protection in the immunised animals. Animals were vaccinated at day 0, 14 and 28 with 15 ug of immunoconjugate without any adjuvant and challenged at day 43 with a lethal dose of infectious virus carrying the H5 gene from the avian pandemic strain. The immunised animals showed marked protection against infection with a 67% survival rate and a significant reduction of viral titres in the lungs of the immunised animals, including the absence of detectable virus in some of the survivors. Again, the immunised animals showed no detectable HAI titres before challenge but the immunised animals did show HAI titres against H5 after viral challenge albeit lower than the 4-fold increase in titre required for the licensing of the annual influenza vaccine. It thus appears that the ability of the Fc immunoconjugates to induce a cellular immune response is sufficient to protect against avian influenza and the use of rapid recombinant DNA technology to produce the immunoconjugates should have particular utility in the production of a vaccine against an emergent pandemic virus.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A fusion protein comprising one or more antigenic polypeptides and an Fc receptor binding polypeptide which comprises a CH2 constant domain of a human IgG immunoglobulin, which binds to an Fc receptor with a binding affinity sufficient to cause internalisation of the bound Fc receptor, wherein the CH2 constant domain contains an asparagine residue at a position equivalent to residue 297 of the CH2 constant domain of a human IgG immunoglobulin as defined in the Kabat database.

2.-3. (canceled)

4. The fusion protein as claimed in claim 1 wherein the Fc receptor binding polypeptide comprises the CH2 constant domain of human IgG immunoglobulin IgG1 or IgG3.

5. (canceled)

6. The fusion protein as claimed in claim 1 wherein the Fc receptor binding polypeptide comprises the amino acid sequence of SEQ ID NO:1.

7. The fusion protein as claimed in claim 1 wherein the Fc receptor binding polypeptide comprises the amino acid sequence of SEQ ID NO:2.

8.-9. (canceled)

10. The fusion protein as claimed in claim 1 wherein the Fc receptor binding polypeptide and the one or more antigenic polypeptides are joined by a covalent bond.

11. The fusion protein as claimed in claim 1 wherein the Fc receptor binding polypeptide and the one or more antigenic polypeptides are joined by a non-covalent bond.

12. The fusion protein as claimed in claim 1 wherein the Fc receptor binding polypeptide and the one or more antigenic polypeptides are joined by a linker moiety or spacer sequence.

13. The fusion protein as claimed in claim 1 wherein the one or more antigenic polypeptides are selected from the group consisting of a viral polypeptide, a bacterial polypeptide, a fungal polypeptide, and a polypeptide derived from a parasite.

14. The fusion protein as claimed in claim 1 wherein the one or more antigenic polypeptides are derived from a non-pathogenic disease selected from the group consisting of a tumour specific antigen, an autoimmune disease and a neurodegenerative disease.

15. The fusion protein as claimed in claim 13 wherein the one or more antigenic polypeptides are viral polypeptides and the virus is HIV, Hepatitis B, or Hepatitis-C.

16. The fusion protein as claimed in claim 13 wherein the one or more antigenic polypeptides are viral polypeptides and the virus is Influenza type A.

17-18. (canceled)

19. An immunogenic composition comprising the fusion protein according to claim 1.

20. The immunogenic composition as claimed in claim 19 further comprising at least one adjuvant.

21-28. (canceled)

29. A method for inducing an immune response in a subject, the method comprising the steps of:
providing a fusion protein according to claim 1, and
administering a therapeutically effective amount of said fusion protein to a subject in which the induction of the immune response against said one or more antigenic polypeptide polypeptides or a fragment thereof provided within the fusion protein is desired.

30. The method as claimed in claim 29 wherein the immune response is for the treatment of infection by a pathogen causative of a pathogenic disease, and
the one or more antigenic polypeptides of the fusion protein are derived from the pathogen or a product derived from the pathogen.

31. The method as claimed in claim 30 wherein one or more antigenic polypeptide polypeptides are derived from a bacterial pathogen or a viral pathogen.

32. The method as claimed in claim 29 wherein the immune response is for the treatment of a non-pathogenic disease condition,
and the one or more antigenic polypeptides of the fusion protein are specific to the non-pathogenic disease condition.

33. The method as claimed in claim 32 wherein the one or more non-pathogenic antigenic polypeptides are selected from the group consisting of a tumour specific polypeptide, a polypeptide which is specific to a neurodegenerative disease and a polypeptide which is specific to an autoimmune disease.

34. The method as claimed in claim 29 wherein the immune response is a cytotoxic T lymphocyte (CTL) response.

35. The method as claimed in claim 29 wherein the subject is a mammal.

36-54. (canceled)
